(19) United States
(12) Patent Application Publication
(10) Pub. No.: US 2015/0306251 A1
(43) Pub. Date: Oct. 29, 2015

(54) ALBUMIN COMPOSITION

(71) Applicant: NOVOZYMES BIOPHARMA UK LIMITED, Nottingham Nottinghamshire (GB)

(72) Inventor: Mark Christopher Perkins, Nottingham (GB)

(21) Appl. No.: 14/418,019

(22) PCT Filed: Aug. 1, 2013
(86) PCT No.: PCT/EP2013/066235

§ 371 Date: Jan. 28, 2015

Related U.S. Application Data
(60) Provisional application No. 61/678,389, filed on Aug. 1, 2012.

(30) Foreign Application Priority Data
Aug. 1, 2012 (EP) 12178876.4

(51) Int. Cl. A61K 49/00; A61K 47/32; A61K 47/42; A61K 47/36; A61K 9/00; A61K 9/08

(52) U.S. Cl. CPC A61K 49/0004; A61K 9/0019; A61K 9/08; A61K 47/42; A61K 47/36; A61K 47/32

(57) ABSTRACT

The present invention relates to use of albumin and a viscosity modifier, particularly a viscosity increasing agent, in a placebo.

### REFERENCE TO SEQUENCE LISTING

This application contains a Sequence Listing in computer readable form. The computer readable form is incorporated herein by reference.

### FIELD OF THE INVENTION

The present invention relates to controlled viscosity albumin compositions. The compositions may be used as placebos. The invention also relates to methods for producing such compositions and to a method for carrying out a clinical trial using such compositions.

### BACKGROUND OF THE INVENTION

A placebo is a medicine that is ineffective but may help to relieve a condition because a recipient patient has faith in its powers. For example, the US Food and Drug Administration (FDA) defines a placebo as “an inactive substance that may resemble an active agent but has no medical value”. When new drugs are tested against placebos in clinical trials, a drug's effect is compared with the placebo response which occurs even in the absence of any pharmacologically active substance in the placebo. In order for new drugs to be appropriately tested, it is important that the patients undergoing the clinical trial and the person analyzing the trial do not know which patients receive the test drug and which patients receive the placebo, i.e. a ‘blind trial’. A trial in which the person receiving the product does not know which product is drug and which product is placebo but where the person delivering the product (e.g. a doctor) or the person analyzing the trial does know which product is drug and which product is placebo is known as a ‘single blind trial’. A trial in which the person receiving the product and the person delivering the product (e.g. a doctor) or the person analyzing the trial do not know which product is drug and which product is placebo is known as a ‘double blind trial’. Therefore, typically, the drug and placebo are provided under coded names.

Typically, a placebo is identical to the test drug with the exception that the pharmacologically active ingredient is absent. However, when the pharmacologically or active ingredient has certain properties, it may be possible for the patient and/or person providing the drug or placebo to identify whether the product being provided is a drug or a placebo. For example, if the product being tested is a liquid for injection and the pharmacologically active ingredient alters the viscosity of the liquid, the patient and doctor will notice the difference between a high viscosity product (such as a test drug) which will be relatively difficult to administer and relatively painful to receive and a low viscosity product (such as a placebo) which will be relatively easy to administer and relatively painless to receive. Examples of liquid placebos include water for injection, normal saline (0.9% (w/v) NaCl), a pharmaceutically acceptable buffer, and 0.1% (w/v) albumin solution. Monoclonal antibodies are an example of drugs that are administered by injection but increase the viscosity of the liquid to an extent which may be detectable by the patient and/or the doctor.

Therefore, what is required is a placebo which more closely mimics the viscosity of a liquid drug composition.

### SUMMARY OF THE INVENTION

The invention provides controlled viscosity albumin compositions useful, e.g., for placebo applications; a method for producing such a composition and a method for carrying out a clinical trial using such a composition.

### BRIEF DESCRIPTION OF DRAWINGS

FIG. 1 shows the relationship between albumin viscosity (dynamic, mPa·s) and albumin concentration (% w/w). FIG. 1a plots viscosity on a linear scale, FIG. 2b plots the viscosity data on a logarithmic scale.

FIG. 2. shows the effect of hyaluronic acid concentration (% w/w) on the viscosity (dynamic, mPa·s) of a 5% (w/w) albumin solution. FIG. 2a plots viscosity on a linear scale, FIG. 2b plots the viscosity data on a logarithmic scale. FIG. 2a was generated using SigmaPlot software and provides the line of best fit and upper and lower 95% prediction intervals.

FIG. 3. shows the effect of hyaluronic acid concentration (% w/w) on the viscosity (dynamic, mPa·s) of a 10% (w/w) albumin solution. FIG. 3a plots viscosity on a linear scale, FIG. 3b plots the viscosity data on a logarithmic scale. FIG. 3a was generated using SigmaPlot software and provides the line of best fit and upper and lower 95% prediction intervals.

FIG. 4. shows the effect of hydroxypropyl methylcellulose (HPMC) concentration (% w/w) on the viscosity (dynamic, mPa·s) of a 5% (w/w) albumin solution. FIG. 4 was generated using SigmaPlot software and provides the lines of best fit and upper and lower 95% prediction intervals.

FIG. 5. shows the effect of polyvinylpyrrolidone (PVP) concentration (% w/w) on the viscosity (dynamic, mPa·s) of a 5% (w/w) albumin solution. FIG. 5 was generated using SigmaPlot software and provides the lines of best fit and upper and lower 95% prediction intervals.

FIG. 6. shows the effect of PVP concentration (% w/w) on the viscosity (dynamic, mPa·s) of a 10% (w/w) albumin solution. FIG. 6 was generated using SigmaPlot software and provides the lines of best fit and upper and lower 95% prediction intervals.

### DETAILED DESCRIPTION OF THE INVENTION

A first aspect of the invention provides a liquid composition comprising or consisting of albumin and a viscosity modifier wherein the composition has a dynamic viscosity from 1 to 250 mPa·s at 25° C. It is preferred that the liquid composition does not contain a pharmacologically effective amount of a pharmacologically active ingredient, such as a drug. It is more preferred that the composition does not contain a pharmacologically active ingredient.

The dynamic viscosity of the liquid composition may be from 1 to 250 mPa·s when measured at 20 to 25° C., most preferably 25° C. More preferably the dynamic viscosity of the liquid composition is from about 1, 5, 10, 20, 30, 40, 50, 75, 100, 125, 150, 160, 170, 180, 190, 200, 225 mPa·s to about 5, 10, 20, 30, 40, 50, 75, 100, 125, 150, 160, 170, 180, 190, 200, 225, 250 mPa·s when measured at 20 to 25° C., preferably 25° C. A dynamic viscosity of at least 5, 10, 20, 30 is preferred, preferably at least 30 mPa·s. A dynamic viscosity of from about 40 to about 160 mPa·s when measured at 25° C. is particularly preferred. In contrast, when measured at 25° C., water has a dynamic viscosity of about 0.89 mPa·s and a 5% (w/w) albumin solution has a dynamic viscosity of about 1.2 mPa·s (Table 2).

It is preferred that the composition contains more albumin (w/w) than viscosity modifier (w/w). Therefore, it is preferred that the liquid composition of albumin contains at least 51 parts albumin to 49 parts viscosity modifier. The liquid composition of albumin may contain albumin and viscosity modifier in a ratio of from 1 part viscosity modifier to 5 parts of albumin, to 1 part viscosity modifier to 2500 parts albumin such as from about 1:5, 1:6, 1:7, 1:8, 1:9, 1:10, 1:15, 1:20, 1:25, 1:30, 1:35, 1:40, 1:45, 1:50, 1:100, 1:250, 1:500, 1:750, 1:1000, 1:1500, 1:2000 to about 1:6, 1:7, 1:8, 1:9, 1:10, 1:15, 1:20, 1:25, 1:30, 1:35, 1:40, 1:45, 1:50, 1:100, 1:250, 1:500, 1:750, 1:1000, 1:1500, 1:2000, 1: 2500 (where the ratio x:y is viscosity modifier:albumin).

The albumin may be present in the liquid composition at from about 5 to about 25% (w/w), such as at from about 5, 6, 7, 8, 9, 10, 11, 12, 13, 14, 15, 16, 17, 18, 19, 20, 21, 22, 23, or 24% to about 6, 7, 8, 9, 10, 11, 12, 13, 14, 15, 16, 17, 18, 19, 20, 21, 22, 23, 24 or 25% (w/w). It is preferred that albumin is present at from 1 to 20%, 10 to 20%, 5 to 15% or 10 to 15% (w/w).

The term ‘viscosity modifier’ means a molecule, such as a macromolecule, that can increase or decrease the viscosity of a liquid composition of albumin, for example increase the viscosity of a liquid composition of albumin by up to 250-fold when present at concentrations of 0.01 to 10% (w/w). For example, the viscosity modifier may increase the viscosity of the liquid formulation of albumin by from about 2, 5, 10, 25, 50, 75, 100, 125, 150, 175, 200, 225 to about 5, 10, 25, 50, 75, 100, 125, 150, 175, 200, 225, 250 fold. The modifier may be present at from 0.01 to 10% (w/w) such as from about 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9 to about 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10% (w/w). It is more preferred that the viscosity modifier is present at from 0.01 to 1%, 0.05 to 5%, 0.05 to 1% or 0.05 to 0.5% (w/w).

The viscosity modifier may be selected from those that increase or those that decrease the viscosity of the liquid composition of albumin. A viscosity modifier that increases the viscosity is preferred. Viscosity modifiers may or may not be selected from glycosaminoglycans (also known as mucopolysaccharides), such as anionic non-sulfated glycosaminoglycans for example hyaluronic acid (HA); extracellular matrix extracts including but not limited to collagen, atelocollagen, protamine; polyamino acids including but not limited to polyarginine, polyornithine; gelatin, amylopectin, maltodextrin, dextran, glycogen, chondroitin, cellulose, cellulose derivatives including, but not limited to, hydroxypropyl methylcellulose (HPMC, also known as hypromellose), hydroxyethyl cellulose, carboxymethyl cellulose or its salts such as sodium carboxymethylcellulose, and the like; cyclodextrins, polyesters, including but not limited to polylactic acids (PLA); polyorthoesters, sulfate, dermatan sulfate, hydrophobic polymers such as polyvinyl alcohols such as polyvinylpyrrolidones (PVP), as well as mixtures containing one or more (several) grades or molecular weight of PVP, polyethylene glycols, polyethyleneoxide, polysaccharides including but not limited to carrageenan, guar gum, alginates, xanthan gum; carbomers, polyvinyl alcohol.

Anionic non-sulfated glycosaminoglycans such as hyaluronic acid; cellulose or cellulose derivatives such as HPMC; and polyvinyl alcohols such as PVP are particularly preferred.

Hyaluronic acid with an average molecular weight of from about 500 to about 2500 is preferred, such as an average molecule weight of from about 500, 600, 700, 800, 900, 1000, 1200, 1400, 1600, 1800, 2000, 2200, 2400 to about 600, 700, 800, 900, 1000, 1200, 1400, 1600, 1800, 2000, 2200, 2400, 2500 KDa. An average molecular weight of from about 600 to about 1100, for example from about 800 to about 1000 or from about 800 to about 900, such as about 850 KDa is preferred.

HPMC with a weight average molecular weight (Mw) of from about 1000 to 150 000 is preferred, such as an average molecular weight of from about 1000 to about 10 000. A weight average molecular weight of from about 2600 to 5600 is preferred (such as measured in a 2% solution in water at 20° C.).

PVP with a Mw of from about 100 000 to about 500 000, such as from about 250 000 to about 450 000 is preferred, more preferably from about 300 000 to about 400 000, most preferably about 360 000.

When the viscosity modifier comprises a polysaccharide, such as an anionic non-sulfated glycosaminoglycans e.g. HA, about 0.2 to 1.2% (w/w) viscosity modifier is preferred. When the viscosity modifier comprises a cellulose or cellulose derivative such as HPMC, e.g. HPMC with a Mw of 2600 to 5600 (such as measured in a 2% solution in water at 20° C.) about 0.2 to 0.6% (w/w) viscosity modifier is preferred. When the viscosity modifier comprises a polyvinyl alcohol such as PVP e.g. PVP with an Mw of 360 000 (K-value 80 to 100), about 1 to 3% (w/w) viscosity modifier is preferred.

It is preferred that the glycosaminoglycan does not have a pharmacological activity at the concentration used in the composition according to the invention, for example it is preferred that the glycosaminoglycan is not heparin or chondroitin sulfate.

A composition according to the invention may comprise one or more (several) viscosity modifiers.

It is particularly preferred that the composition does not comprise the pharmacologically active ingredient which is the ingredient being tested e.g. in a clinical trial using the placebo of the present invention and a test composition.

The liquid composition of albumin may comprise from about 1 to about 250% (w/w) albumin such as from about 1, 2, 3, 4, 5, 6, 7, 8, 9, 10, 11, 12, 13, 14, 15, 16, 17, 18, 19, 20, 21, 22, 23, 24 to about 2, 3, 4, 5, 6, 7, 8, 9, 10, 11, 12, 13, 14, 15, 16, 17, 18, 19, 20, 21, 22, 23, 24, 25% (w/w). Preferred compositions comprise 1 to 20%, 10 to 20%, 5 to 15% or 10 to 15% (w/w) albumin.

For a liquid composition comprising albumin at 10% (w/w) or higher, it is preferred that the viscosity modifier is not a cellulose or cellulose derivative, it is more preferred that the viscosity modifier is not HPMC and it is particularly preferred that the viscosity modifier is not HPMC with an Mw of 2600 to 5600 (such as measured in a 2% solution in water at 20° C.).

The albumin may be any albumin, fragment or variant thereof. It is preferred that the albumin has at least 70% identity to human serum albumin (SEQ ID NO: 2), more preferably 75, 80, 85, 90, 95, 96, 97, 98, 98.5, 99, 99.5, 99.6, 99.7, 99.8, 99.9% identity human serum albumin. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

SEQ ID NO: 2 may be encoded, for example, by the polynucleotide sequence of SEQ ID NO: 1. It is preferred that a fragment of albumin is at least 300, 350, 400, 450, 500 or 550 amino acids long. The albumin may or may not be genetically fused to a fusion partner. The albumin may or may not be conjugated, e.g. chemically conjugated, to a conjugation partner. A fusion partner may be any non-albumin polypeptide such as a pharmacologically or active polypeptide or a polypeptide useful in diagnosis or imaging. A conjugation partner may be any chemical or non-albumin polypeptide, such a pharmacologically active chemical or a chemical or polypeptide useful in diagnosis or imaging.

It is preferred that the albumin has the same and/or very similar tertiary structure as human serum albumin (HSA) or one or more (several) of HSA domains I, II or III and has similar properties to HSA or the relevant domains. Similar tertiary structures are, for example, the structures of the albumins from the species mentioned under parent albumin. Some of the major properties of albumin are i) its ability to regulate plasma volume (oncotic activity), ii) a long plasma half-life of around 19 days±5 days, iii) binding to FcRn, v) ligand-binding, e.g. binding of endogenous molecules such as acidic, lipophilic compounds including bilirubin, fatty acids, hemin and thyroxine (see also Table 1 of Kragh-Hansen et al, 2002, Biol. Pharm. Bull. 25, 695, hereby incorporated by reference), iv) binding of small organic compounds with acidic or electronegative features e.g. drugs such as warfarin, diazepam, ibuprofen and paclitaxel (see also Table 1 of Kragh-Hansen et al, 2002, Biol. Pharm. Bull. 25, 695, hereby incorporated by reference). Not all of these properties need to be fulfilled to in order to characterize a protein or fragment as an albumin. If a fragment, for example, does not comprise a domain responsible for binding of certain ligands or organic compounds the variant of such a fragment will not be expected to have these properties either.

The albumin may be a serum-derived or a recombinant product. For example, the albumin may be obtained from human serum albumin or may be obtained from a recombinant host such as a yeast, e.g. Saccharomyces cerevisiae.

For a 5% albumin composition, a composition of desired viscosity may be prepared by using the equations:

Curve of best fit: y=−2.658+3.348*EXP(6.423*x)

Lower prediction interval: y=−3.943+3.383*EXP(6.399*x)

Upper prediction interval: y=−1.374+3.314*EXP(6.446*x)

where y=dynamic viscosity in mPa·s and x=concentration (% w/w) of HA such as with an average molecular weight of from about 600 to about 1100, for example from about 800 to about 1000, such as about 850 KDa.

It is preferred that the composition lies between the area defined by the lower prediction interval and the upper prediction interval. ‘Between’ includes on the intervals. The composition may, more preferably, comply with the curve of best fit.

Alternatively, or additionally, for a 5% albumin composition, a composition of desired viscosity may be prepared using the equations:

Curve of best fit: y=−1.3028+2.2132*EXP(5.1690*x)

Lower prediction interval: y=−3.6984+2.23290*EXP(5.0815*x)

Upper prediction interval: y=1.0836+2.1045*EXP(5.2558*x)

where y=dynamic viscosity in mPa·s and x=concentration (% w/w) of a cellulose or cellulose derivative such as HPMC, e.g. HPMC with an Mw of 1000 to 10 000, preferably from 2600 to 5600 (such as measured in a 2% solution in water at 20° C.).

It is preferred that the composition lies between the area defined by the lower prediction interval and the upper prediction interval. ‘Between’ includes on the intervals. The composition may, more preferably, comply with the curve of best fit.

Alternatively, or additionally, for a 5% albumin composition, a composition of desired viscosity may be prepared using the equations:

Curve of best fit: y=−2.3149+3.6315*EXP(0.5844*x)

Lower prediction interval: y=−2.6554+3.7042*EXP(0.5788*x)

Upper prediction interval: y=−1.9763+3.5605*EXP(0.5899*x)

where y=dynamic viscosity in mPa·s and x=concentration (% w/w) of a polvinvyl alcohol such as PVP, such as PVP with Mw=250 000 to 500 000, preferably 300 000 to 400 000, most preferably Mw=360 000.

It is preferred that the composition lies between the area defined by the lower prediction interval and the upper prediction interval. ‘Between’ includes on the intervals. The composition may, more preferably, comply with the curve of best fit.

For a 10% albumin composition, a composition of desired viscosity may be prepared by using the equations:

Curve of best fit: y=−2.853+3.815*EXP(6.501*x)

Lower prediction interval: y=−4.736+3.864*EXP(6.472*x)

Upper prediction interval: y=−0.971+3.768*EXP(6.531*x)

where y=dynamic viscosity in mPa·s and x=concentration (% w/w) of HA such as with an average molecular weight of from about 600 to about 1100, for example from about 800 to about 1000, such as about 850 KDa.

It is preferred that the composition lies between the area defined by the lower prediction interval and the upper prediction interval. ‘Between’ includes on the intervals. The composition may, more preferably, comply with the curve of best fit.

Alternatively, or additionally, for a 10% albumin composition, a composition of desired viscosity may be prepared using the equations:

Curve of best fit: y=−2.8401+4.3501*EXP(0.5607*x)

Lower prediction interval: y=−3.4081+4.4819*EXP(0.5525*x)

Upper prediction interval: y=−2.2770+4.2230*EXP(0.5689*x)

where y=dynamic viscosity in mPa·s and x=concentration (% w/w) of a polvinvyl alcohol such as PVP, such as PVP with Mw=250 000 to 500 000, preferably 300 000 to 400 000, most preferably Mw=360 000.

It is preferred that the composition lies between the area defined by the lower prediction interval and the upper prediction interval. ‘Between’ includes on the intervals. The composition may, more preferably, comply with the curve of best fit.

850 KDa HA, i.e. HA with an average molecular weight of 850 KDa (specification is from 0.6 to 1.1 MDa), is available, for example, from Novozymes Biopharma DK A/S. HPMC with Mw of 2600 to 5600 is available from Sigma-Aldrich. PVP with Mw of 360 000 is available from Sigma-Aldrich.

Therefore, in one embodiment, the invention provides compositions comprising about 5% or about 10% albumin and which have a HA content and dynamic viscosity which lie on or between the lower and upper prediction intervals as described above. In a further embodiment, the invention provides compositions comprising about 5% or about 10% albumin and which have a PVP content and dynamic viscosity which lie on or between the lower and upper prediction intervals as described above. In another embodiment, the invention provides provides compositions comprising about 5% albumin and which have a HPMC content and dynamic viscosity which lie on or between the lower and upper prediction intervals as described above According to one embodiment, the invention also provides compositions which comply with the curve of best fit for about 10% albumin content or for about 5% albumin content for compositions comprising HA, PVP and/or HPMC as the viscosity modifier.

The skilled person can determine similar equations for other concentrations of HA and other viscosity modifiers. For example, statistics software such as SigmaPlot version 11 (build 11.0.0.75, Systat Software Inc., San Jose Calif. USA) may be used to fit a series of data for a given concentration of albumin (% w/w) with varying HA content (% w/w) and dynamic shear. The software may be used to fit the data, for example using single, 3 parameter exponential growth (y=y0+a*EXP(b*x)) to derive best fit line and the 95% prediction interval. The 95% prediction interval is understood by the skilled person as the region of uncertainties in predicting the response for a single additional observation.

The liquid composition of albumin may be a suspension and/or a solution. The albumin and the viscosity modifier may be suspended or dissolved in any suitable liquid or diluent, such as a pharmaceutically acceptable or physiologically acceptable liquid which may or may not be or comprise an excipient, carrier or stabilizer. The phrases “pharmaceutically acceptable” or “physiologically acceptable” refer to molecular entities and compositions that do not produce adverse, allergic, toxic, or other untoward reactions when administered to a human or an animal.

As used herein, “pharmaceutically acceptable”, or “physiologically acceptable” liquids include any and all solvents and dispersion media. Examples of liquids or diluents include buffers such as phosphate, citrate, and other organic acids; antioxidants including ascorbic acid. Aqueous liquids or diluents are preferred, such as water (e.g. water for injection), saline such as normal saline 0.9% NaCl (w/v), or an aqueous pH buffered solution.

The liquid composition of albumin preferably has a pH of about 4 to about 8, preferably from about 5 to about 7.5 such as from about 5.0, 5.25, 5.5, 5.75, 6.0, 6.25, 6.5, 6.75 to 5.25, 5.5, 5.75, 6.0, 6.25. 6.5, 6.75, 7.0, or 7.25 to about 5.25, 5.5, 5.75, 6.0, 6.25. 6.5, 6.75 to 5.25, 5.5, 5.75, 6.0, 6.25. 6.5, 6.75, 7.0, 7.25, or 7.5.

The liquid composition of albumin may or may not contain a stabilizer, such as one or more (several) selected from fatty acids such as octanoate; detergents such as non-ionic surfactants such as a polysorbate, e.g. polysorbate 80 or polysorbate 20; amino acids such as N-acetyl tryptophan, histidine, lysine, arginine, glycine, glutamine, asparagine or an L-hydrochloride thereof; or sugars such as α,α-trehalose dehydrate, sucrose; sugar forming alcohols such as sugar alcohols such as mannitol or sorbitol; carbohydrates including glucose, mannose, or dextrins. Octanoate may be present at from about 4 to about 40 mM, such as about 5 to about 35 mM, about 4 to about 12 mM, about 30 to about 35 mM, preferably about 32 mM. A polysorbate, such as polysorbate 80, may be present at from about 10 to about 50 mg/L, preferably about 15 mg/L.

The liquid composition of albumin may or may not contain a salt, such as a sodium salt. The sodium ion concentration may be from about 120 to about 160 mM, preferably about 145 mM. The polymer content of the albumin component may be less than or equal to 1.0% (w/v).

The liquid composition of albumin may contain less than or equal to 0.15 micrograms of host cell protein per gram of albumin if, for example, the albumin is derived from a recombinant source such as a yeast such as S. cerevisiae. The liquid composition of albumin may contain less than or equal to 0.30% (w/w) of Concanavilin A-bound albumin relative to unbound albumin. The liquid composition of albumin may contain less than or equal to 0.5 micrograms of nickel per gram of albumin. The liquid composition of albumin may contain less than or equal to 0.01 millimoles of potassium per gram of protein.

It is preferred that the liquid composition of albumin contains less than about 50 ppm divalent cations such as magnesium and/or calcium, more preferably less than about 40, 20, 30, 10, 5 ppm divalent cations.

It is preferred that the liquid composition of albumin has a similar color to a pharmaceutical test composition, such as the pharmaceutical test composition to which it is being compared. Preferably, the liquid composition of albumin is visually indistinguishable from the pharmaceutical test composition to which it is being compared. For example, the peak absorbance wavelength of the pharmaceutical test composition may be identified by spectrophotometry, the placebo may then be spectrophotometrically analyzed at the same wavelength. An absorbance that is within 20%, preferably 10% most preferably 5% of the pharmaceutical test composition is preferred. In addition of pharmaceutical test composition and the placebo may be measured at 280 nm and at 350 nm. An absorbance score may be determined:

Absorbance score=A₃₅₀×100/A₂₈₀

Absorbance at A₃₅₀ is measured to detect pigment and A₂₈₀ is measured to detect protein. It is preferred that absorbance score of the placebo is from 80% to 120% of the absorbance score of the pharmaceutical test composition. For example, the absorbance score of the placebo may be from about 80, 85, 90, 95, 100, 105, 110, 115 to about 85, 90, 95, 100, 105, 110, 115, 120% of the absorbance score of the pharmaceutical test composition. Preferably, the absorbance score of the placebo is from about 90 to about 110% of the absorbance score of the pharmaceutical test composition.

It is preferred that the absorbance is measured by adjusting (e.g. diluting) the placebo sample so that albumin is present at 1 mg/mL in water such as laboratory grade water e.g. sterile water or MilliQ water and adjusting the test sample by the same factor. Absorbance is preferably measured in 1.5 mL cuvettes, e.g. plastic UV compatible cuvettes (e.g. Plastibrand, Fisher Scientific UK Ltd, Loughborough, UK, catalog number CXA-205-110E). UV compatible means that the cuvettes are made from UV transparent material. A suitable spectrophotometer is Varian Cary 50 UV-VIS (Agilent Technologies UK Ltd, Wokingham, UK).

The color of the liquid composition of albumin may or may not be adjusted so that it is visually indistinguishable from the test pharmaceutical composition. For example, one or more (several) colored components such as pigments or dyes or proteins such as albumin may be added. Alternatively or in addition, the color of the test pharmaceutical composition may or may not be adjusted so that it is visually indistinguishable from the liquid composition of albumin. For example, one or more (several) colored components such as pigments or dyes or proteins such as albumin may be added.

The liquid composition of albumin may or may not contain antibacterial or antifungal agents, isotonic agents, absorption delaying agents, low molecular weight (less than about 10 residues) polypeptide; chelating agents such as EDTA; salt-forming counter-ions such as sodium.

The liquid composition of albumin may or may not comprise a pharmacologically active compound. Since the composition is in one embodiment useful as a placebo, it is preferred that the composition does not contain a pharmacologically active compound. However, since the composition may be useful to compare a combination therapy (e.g. pharmacologically active compound A and pharmacologically active compound B) with single therapies (e.g. pharmacologically active A in the absence of pharmacologically active compound B), the composition may contain one or more (several) pharmacologically active compounds. Therefore, a placebo according to the invention may or may not include a pharmacologically active composition.

The pharmaceutical test composition may or may not comprise an antibody such as a monoclonal antibody. The pharmacologically active compound may or may not comprise an antibody such as a monoclonal antibody. The antibody may or may not be a murine antibody, a human antibody, a humanized antibody, a chimeric antibody or an antibody fragment. The antibody may or may not have a target selected from: a cluster of differentiation (CD) marker such as CD20, CD25, CD3 or CD52; a component of the complement pathway such as Complement C5; a growth factor such as VEGF or VEGF-A; a growth factor receptor such as epidermal growth factor receptor (EGFR), an integrin such as GPIIb/IIIa or VLA-4; HER-2; an immunoglobulin such as IgE; an interleukin such as IL-1β (IL-1 beta), IL6, IL-12 or IL-23; RANKL; a viral protein such as RSV F protein; a tumor necrosis factor such as TNFα. The pharmaceutical test composition may or may not include monoclonal antibodies described by the Animal Cell Technology Industrial Platform (6 Jan. 2011) as shown in Table 1 (below).

TABLE 1
Monoclonal antibodies approved by the FDA.

| Trade | | | | | | Therapeutic |
|---|---|---|---|---|---|---|
| Name | INN¹ | Company | Target | Type | Approval² | indications³ |
| ReoPro ® | abciximab | Centocor | GPIIb/IIIa | chimeric | 1994 | High risk |
| | | Ortho Biotech | | | | angioplasty |
| | | (Johnson & | | | | |
| | | Johnson) and | | | | |
| | | Eli Lilly | | | | |
| Humira ® | adalimumab | Abbott | TNFα | human | 2002 | Rheumatoid |
| | | | | | | arthritis; |
| | | | | | | Juvenile |
| | | | | | | idiopathic |
| | | | | | | arthritis; |
| | | | | | | Psoriatic |
| | | | | | | arthritis; |
| | | | | | | Ankylosing |
| | | | | | | spondylitis; |
| | | | | | | Crohn's |
| | | | | | | disease; Plaque |
| | | | | | | psoriasis |
| Campath ® | alemtuzumab | Millennium | CD52 | humanized | 2001 | B-cell chronic |
| | | Pharmaceuticals | | | | lymphocytic |
| | | and | | | | leukemia |
| | | Genzyme | | | | |
| Simulect ® | basiliximab | Novartis | CD25 | chimeric | 1998 | Transplantation |
| | | | | | | rejection |
| Avastin ® | bevacizumab | Genentech | VEGF | humanized | 2003 | Metastatic |
| | | (Roche) | | | | colorectal |
| | | | | | | cancer; Non- |
| | | | | | | small cell lung |
| | | | | | | cancer; |
| | | | | | | Metastatic |
| | | | | | | breast cancer; |
| | | | | | | Glioblastoma |
| | | | | | | multiforme; |
| | | | | | | Metastatic renal |
| | | | | | | cell carcinoma |
| Ilaris ® | canakinumab | Novartis | IL-1β | human | 2009 | Cryopyrin- |
| | | | | | | associated |
| | | | | | | periodic |
| | | | | | | syndromes, |
| | | | | | | including familial |
| | | | | | | cold |
| | | | | | | autoinflammatory |
| | | | | | | syndrome and |
| | | | | | | Muckle-Wells |
| | | | | | | syndrome |
| Cimzia ® | certolizumab | UCB | TNFa | humanized | 2008 | Crohn's disease |
| | pegol | | | antibody | | Rheumatoid |
| | | | | fragment | | arthritis |
| Erbitux ® | cetuximab | ImClone (Eli | EGFR | chimeric | 2004 | Head and neck |
| | | Lilly), Merck | | | | cancer; |
| | | Serono and | | | | Colorectal |
| | | BMS | | | | cancer |
| Zenapax ® | daclizumab | Roche | CD25 | humanized | 1997 | Transplantation |
| | | | | | | rejection |
| Prolia ® | denosumab | Amgen | RANKL | human | 2010 | Postmenopausal |
| Xgeva ® | | | | | | osteoporosis; |
| | | | | | | Prevention of |
| | | | | | | SREs in |
| | | | | | | patients with |
| | | | | | | bone |
| | | | | | | metastases |
| | | | | | | from solid |
| | | | | | | tumours |
| Soliris ® | eculizumab | Alexion | Complement | humanized | 2007 | Paroxysmal |
| | | Pharmaceuticala | C5 | | | nocturnal |
| | | | | | | hemoglobinuria |
| Simponi ® | golimumab | Centocor | TNFa | human | 2009 | Rheumatoid |
| | | Ortho Biotech | | | | arthritis; |
| | | (Johnson & | | | | Psoriatic |
| | | Johnson) | | | | arthritis; |
| | | | | | | Ankylosing |
| | | | | | | spondylitis |
| Zevalin ® | ibritumomab | Biogen Idec | CD20 | | 2002 | Non-Hodgkin's |
| | tiuxetan | | | | | lymphoma |
| Remicade ® | infliximab | Centocor | TNFα | chimeric | 1998 | Crohn's |
| | | Ortho Biotech | | | | disease; |
| | | (Johnson & | | | | Ulcerative |
| | | Johnson) | | | | colitis; |
| | | | | | | Rheumatoid |
| | | | | | | arthritis; |
| | | | | | | Ankylosing |
| | | | | | | spondylitis |
| | | | | | | Psoriatic |
| | | | | | | arthritis; |
| | | | | | | Plaque psoriasis |
| Orthoclone | muromonab- | Centocor | CD3 | murine | 1986 | Transplantation |
| OKT3 ® | DC3 | Ortho Biotech | | | | rejection |
| | | (Johnson & | | | | |
| | | Johnson) | | | | |
| Tysabri ® | natalizumab | Biogen Idec | VLA-4 | humanized | 2004 | Multiple |
| | | and Elan | | | | sclerosis |
| | | | | | | (relapsing); |
| | | | | | | Crohn's disease |
| Arzerra ® | ofatumumab | Genmab and | CD20 | human | 2009 | Chronic |
| | | GSK | | | | lymphocytic |
| | | | | | | leukemia |
| Xolair ® | omalizumab | Genentech | IgE | humanized | 2003 | Asthma |
| | | (Roche) and | | | | |
| | | Novartis | | | | |
| Synagis ® | palivizumab | MedImmune | RSV F | humanized | 1998 | Respiratory |
| | | (AZ) | protein | | | syncytial virus |
| Vectibix ® | panitumumab | Amgen | EGFR | human | 2006 | Metastatic |
| | | | | | | colorectal |
| | | | | | | carcinoma |
| Lucentis ® | ranibizumab | Genentech | VEGF-A | humanized | 2006 | Neovascular |
| | | (Roche) | | antibody | | (wet) age- |
| | | | | fragment | | related macular |
| | | | | | | degeneration; |
| | | | | | | Macular edema |
| | | | | | | following retinal |
| | | | | | | vein occlusion |
| Rituxan ® | rituximab | Biogen Idec | CD20 | chimeric | 1997 | Non-Hodgkin's |
| | | and | | | | lymphoma; |
| | | Genentech | | | | Chronic |
| | | (Roche) | | | | lymphocytic |
| | | | | | | leukemia; |
| | | | | | | Rheumatoid |
| | | | | | | arthritis |
| Actemra ® | tocilizumab | Chugai | IL-6 | humanized | 2010 | Rheumatoid |
| | | (Roche) | | | | arthritis |
| Bexxar ® | tositumomab | Corixa and | CD20 | murine | 2003 | Non-Hodgkin's |
| | and | GSK | | | | lymphoma |
| | iodine 131 | | | | | |
| | tositumomab | | | | | |
| Herceptin ® | trastuzumab | Genentech | HER-2 | humanized | 1998 | Breast cancer; |
| | | (Roche) | | | | Metastatic |
| | | | | | | gastric or |
| | | | | | | gastroesophageal |
| | | | | | | junction |
| | | | | | | adenocarcinoma |
| Stelara ® | ustekinumab | Centocor | IL-12 | human | 2009 | Plaque psoriasis |
| | | Ortho Biotech | IL-23 | | | |
| | | (Johnson & | | | | |
| | | Johnson) | | | | |

¹INN: International Non-proprietary Name
²Approval: Year of first approval by US Food and Drug Administration (FDA)
³Therapeutic Indications: Therapeutic indications approved by the FDA

A second aspect of the invention provides a placebo comprising or consisting of the liquid composition of albumin according to the first aspect of the invention. Each of the preferences for the first aspect of the invention also applies to the second aspect of the invention. As described in relation to the first aspect of the invention, a placebo according to the invention may or may not include a pharmacologically active composition.

It is preferred that the placebo has a dynamic viscosity which is from about 70 to about 130% of the dynamic viscosity of the pharmaceutical test composition, for example from about 80, 85, 90, 95, 96, 97, 98, 99, 100, 105, 110, 115 to about 85, 90, 95, 100, 101, 102, 103, 104, 105, 110, 115, 120% of the dynamic viscosity of the pharmacological or pharmaceutical test composition. More preferred, the placebo has a dynamic viscosity from about 95 to about 105% of the pharmacological or pharmaceutical test composition, most preferred about 100%.

It is preferred that the placebo has an injection force profile which is comparable to the injection force profile of the pharmaceutical test composition. An injection force profile includes:
the force required to be imparted on the liquid in order for it to start to flow. This can be referred to as the ‘peak force’;
the time taken from initial application of the force to reaching the peak force, this can be referred to as the ‘time to peak force’;
the ‘rate of force development’ (i.e. ‘peak force’ divided by ‘time to peak force’).

It is preferred that the placebo has a peak force of from about 50 to about 200% of the peak force of the pharmaceutical test composition, such as from about 50, 60, 70, 80, 90, 100, 110, 120, 130, 140, 150, 160, 170, 180, 190 to about 60, 70, 80, 90, 100, 110, 120, 130, 140, 150, 160, 170, 180, 190, 200%.

It is preferred that the placebo has a time to peak force of from about 50 to about 200% of the time to peak force of the pharmaceutical test composition, such as from about 50, 60, 70, 80, 90, 100, 110, 120, 130, 140, 150, 160, 170, 180, 190 to about 60, 70, 80, 90, 100, 110, 120, 130, 140, 150, 160, 170, 180, 190, 200%.

It is preferred that the placebo has a rate of force development of from about 50 to about 200% of the rate of force development of the pharmaceutical test composition, about 50, 60, 70, 80, 90, 100, 110, 120, 130, 140, 150, 160, 170, 180, 190 to about 60, 70, 80, 90, 100, 110, 120, 130, 140, 150, 160, 170, 180, 190, 200%.

It is preferred that the placebo has a peak force of from 1 N to 20 N, such as from about 1, 2, 3, 4, 5, 6, 7, 8, 9, 10, 11, 12, 13, 14, 15, 16, 17, 18, 19 to about 2, 3, 4, 5, 6, 7, 8, 9, 10, 11, 12, 13, 14, 15, 16, 17, 18, 19, 20 N. A peak force of from about 10 to about 15 is preferred.

It is preferred that the injection force profile is determined using a needle with a gauge from 15 to 34 or higher (i.e. narrower diameter), preferably from 20 to 34 or higher. A needle of gauge of about 30 is preferred.

It is preferred that the injection force profile is determined using a placebo volume of from about 1 to about 5 ml. A volume of about 2 ml is preferred.

It is preferred that the injection force profile is determined using an injection rate of about 2 to about 5 ml per minute, preferably about 2 ml per minute.

A third aspect of the invention provides a method for preparing a composition of a desired viscosity comprising suspending albumin and a viscosity modifier in a liquid in a ratio complying with about 1 part viscosity modifier and from about 5 to about 2500 parts albumin. It is preferred that the composition is a liquid composition of albumin according to the first and/or second aspect of the invention. For example:

For a 5% albumin composition, a composition of desired viscosity may be prepared by using the equations:

Curve of best fit: y=−2.658+3.348*EXP(6.423*x)

Lower prediction interval: y=−3.943+3.383*EXP(6.399*x)

Upper prediction interval: y=−1.374+3.314*EXP(6.446*x)

where y=dynamic viscosity in mPa·s and x=concentration (% w/w) of HA such as with an average molecular weight of from about 600 to about 1100, for example from about 800 to about 1000, such as about 850 KDa.

It is preferred that the composition lies between the area defined by the lower prediction interval and the upper prediction interval or on one of these intervals. The composition may, more preferably, comply with the curve of best fit.

For a 10% albumin composition, a composition of desired viscosity may be prepared by using the equations:

Curve of best fit: y=−2.853+3.815*EXP(6.501*x)

Lower prediction interval: y=−4.736+3.864*EXP(6.472*x)

Upper prediction interval: y=−0.971+3.768*EXP(6.531*x)

where y=dynamic viscosity in mPa·s and x=concentration (% w/w) of HA such as with an average molecular weight of from about 600 to about 1100, for example from about 800 to about 1000, such as about 850 KDa.

It is preferred that the composition lies between the area defined by the lower prediction interval and the upper prediction interval or on one of these intervals. The composition may, more preferably, comply with the curve of best fit.

Alternatively, or additionally, for a 5% albumin composition, a composition of desired viscosity may be prepared using the equations:

Curve of best fit: y=−1.3028+2.2132*EXP(5.1690*x)

Lower prediction interval: y=−3.6984+2.23290*EXP(5.0815*x)

Upper prediction interval: y=1.0836+2.1045*EXP(5.2558*x)

where y=dynamic viscosity in mPa·s and x=concentration (% w/w) of a cellulose or cellulose derivative such as HPMC, e.g. HPMC with an Mw of from 1000 to 10 000, preferably from 2600 to 5600 (such as measured in a 2% solution in water at 20° C.).

It is preferred that the composition lies between the area defined by the lower prediction interval and the upper prediction interval. ‘Between’ includes on the intervals. The composition may, more preferably, comply with the curve of best fit.

Alternatively, or additionally, for a 5% albumin composition, a composition of desired viscosity may be prepared using the equations:

Curve of best fit: y=−2.3149+3.6315*EXP(0.5844*x)

Lower prediction interval: y=−2.6554+3.7042*EXP(0.5788*x)

Upper prediction interval: y=−1.9763+3.5605*EXP(0.5899*x)

where y=dynamic viscosity in mPa·s and x=concentration (% w/w) of a polvinvyl alcohol such as PVP, such as PVP with Mw=250 000 to 500 000, preferably 300 000 to 400 000, most preferably Mw=360 000.

It is preferred that the composition lies between the area defined by the lower prediction interval and the upper prediction interval. ‘Between’ includes on the intervals. The composition may, more preferably, comply with the curve of best fit.

Each of the preferences for the first and second aspects of the invention also apply to the third aspect of the invention.

A fourth aspect of the invention provides use, or a method of use, of a composition according to the first, second and/or third aspect of the invention as a placebo. For example, the use or method may include:
(a) providing a pharmaceutical test composition;
(b) providing a placebo composition according to the first, second or third aspect of the invention in which the compound being tested is omitted;
(c) comparing the pharmaceutical test composition and the placebo composition.
   The test may include visual analysis such as comparing the appearance e.g. color. The test may include testing the effectiveness of the pharmaceutical test composition relative to the placebo composition. Therefore, the invention also provides a method of administering to a patient or a method of carrying out a clinical trial comprising:
(a) administering to a first group of patients, a pharmaceutical test composition;
(b) administering to a second group of patients, a placebo which has a viscosity from about 50 to about 250-fold compared to the viscosity of the pharmacological or pharmaceutical test composition, wherein the placebo is a composition according to the first, second and/or third aspect of the invention;
(c) analyzing the effectiveness of the pharmaceutical test composition relative to the placebo; and/or
(d) optionally generating a qualitative or quantitative comparison of the effectiveness of the pharmaceutical test composition and the placebo; and/or
(e) optionally concluding whether or not the pharmaceutical test composition has a clinically significant effect; and/or
(f) optionally using the output of step (d) and/or step (e) to determine whether or not to carry out a subsequent step in the administration to a patient or in the clinical trial.
   The clinical trial is preferably a blind trial, such as a single blind trial or, more preferably, a double blind trial.

The clinical trial may comprise one or more (several) patients such as from 1 to 10 000, 10 to 1000, 10 to 100 or 10 to 50 patients. It is preferred that the placebo-assigned group and the test-assigned group are of similar sizes, for example within 10% of the size of each other.

For example, the effectiveness of a test composition against a particular disease or condition might be determined by calculating relative risk or risk difference (e.g. Chapter 7 of Hackshaw, A. (2009). A Concise Guide to Clinical Trials, Publisher: Wiley; incorporated herein by reference). Relative risk tends to be similar across different populations, indicating the effect of a new intervention, e.g. drug, generally. Relative risk does not usually depend on the underlying rate of disease. Risk difference indicates the effect of a treatment in a particular population. Risk difference takes into account the underlying rate of disease and therefore varies between populations. Risk difference compares the likelihood of a first group of patients, who received a pharmaceutical test composition, of experiencing an adverse event (e.g. catching an infectious disease, developing a condition, not recovering satisfactorily from an existing disease or condition) compared with likelihood of a second group of patients, who received a placebo composition according to the present invention.

As a disease becomes more common, relative risk is not expected to change much but there is expected to be an increase in risk difference and a decrease in the number of patients which must be treated in order to avoid one individual experiencing an adverse event, this is known as the ‘Number Needed to Treat (NNT)’. Therefore, an intervention has a greater effect in a population when a disease is common. As a measure of effectiveness, relative risk is preferred.

Relative risk and/or risk difference and/or NNT can be measured over a desired time frame such as from 1, 2, 4, 8, 24 hours, 2, 3, 4, 5, 6, 7 days, 2, 3, 4 weeks, 1, 2, 3, 4, 5, 6, 7, 8, 9, 10, 11, 12, 15, 18, 21, 24, 36, 48, months to 2, 4, 8, 24 hours, 2, 3, 4, 5, 6, 7 days, 2, 3, 4 weeks, 1, 2, 3, 4, 5, 6, 7, 8, 9, 10, 11, 12, 15, 18, 21, 24, 36, 48, 60 months.

For relative risk, risk difference and/or NNT a confidence interval of 95% is preferred. Calculation of confidence intervals is known to the skilled person. For example, confidence intervals may be calculated using one or more (several) of the tests described in Box 7.9 on page 114, Chapter 7 of Hackshaw, A. (2009). A Concise Guide to Clinical Trials, Publisher: Wiley; incorporated herein by reference).

Relative risk: With regards a pharmaceutical test composition designed to reduce the risk of experiencing an adverse event (e.g. catching an infectious disease, developing a condition, not recovering satisfactorily from an existing disease or condition) a relative risk of less than 0.9, 0.8, 0.7, 0.6, 0.5, 0.4, 0.3, 0.2, 0.1 or 0.05 may be the threshold required to proceed to a subsequent step of a trial or treatment or to determine that a pharmaceutical test composition has a clinically significant effect. For example, a relative risk of 0.4 means that a patient treated with a pharmaceutical test composition has 40% risk of the adverse event compared to a patient given placebo.

Risk difference: With regards a pharmaceutical test composition designed to reduce the risk of experiencing an adverse event (e.g. catching an infectious disease, developing a condition, not recovering satisfactorily from an existing disease or condition), a risk difference of at least 1, 2, 3, 4, 5, 10, 20, 30, 40, 50% may be the threshold required to proceed to a subsequent step of a trial or treatment or to determine that a pharmaceutical test composition has a clinically significant effect. For example, a risk difference of 5% means that for a group of 100 patients treated with a drug there will be 5 fewer patients experiencing an adverse event compared with a group of 100 patients given placebo.

Number Needed to Treat (NNT): With regards a pharmaceutical test composition designed to reduce the risk of experiencing an adverse event (e.g. catching an infectious disease, developing a condition, not recovering satisfactorily from an existing disease or condition), an NNT of at most 5, 10, 20, 25, 50, 100, 200, 250, 500, 1000 may be the threshold required to proceed to a subsequent step of a trial or treatment or to determine that a pharmaceutical test composition has a clinically significant effect. For example, an NNT of 100 means that 100 patients need to be treated with the pharmaceutical test composition to avoid the occurrence of the adverse event, which the pharmaceutical test composition is designed to prevent, in 1 patient.

Each of the preferences for the first, second and third aspects of the invention also apply to the fourth aspect of the invention.

The present invention is further described by the following examples that should not be construed as limiting the scope of the invention.

### EXAMPLES

### Example 1

The viscous properties of liquid formulations comprising albumin (HSA) and/or hyaluronic acid (HA) were studied.

Materials: Recombinant human serum albumin: 35% (w/w) preparation of Recombumin® (Novozymes Biopharma DK A/S); Sodium chloride; Sodium Octanoate; Polysorbate 80; Deionized water (MilliQ water, freshly tapped); Hyaluronic acid: Hyasis® 850 T, (average molecular weight of the batch used was 1000 kDa, i.e. within the specification 0.6 to 1.1 MDa) (Novozymes Biopharma DK A/S).

Equipment: Viscosity was measured using a rheometer (Anton Paar Physica MCR301) at 25° C. using Geometry; C-CC27-T200/SS, CC27.

Liquid compositions were prepared by mixing the required amounts of albumin and/or hyaluronic acid in the following buffer: Buffer: 3000 mL; Sodium Chloride—145 mM (58.44 g/mol); Sodium Octanoate—32 mM (166.19 g/mol); Polysorbate 80—15 mg/L; MilliQ water—q.s. (Table 2a).

TABLE 2a

| Component for 3000 g | Amount, g | |
|---|---|---|
| Sodium Chloride | 25.420 | |
| Sodium Octanoate | 15.950 | |
| Polysorbate 80 | 0.0450 | |
| MilliQ water (to volume) | (to volume) 3000.0 | g |

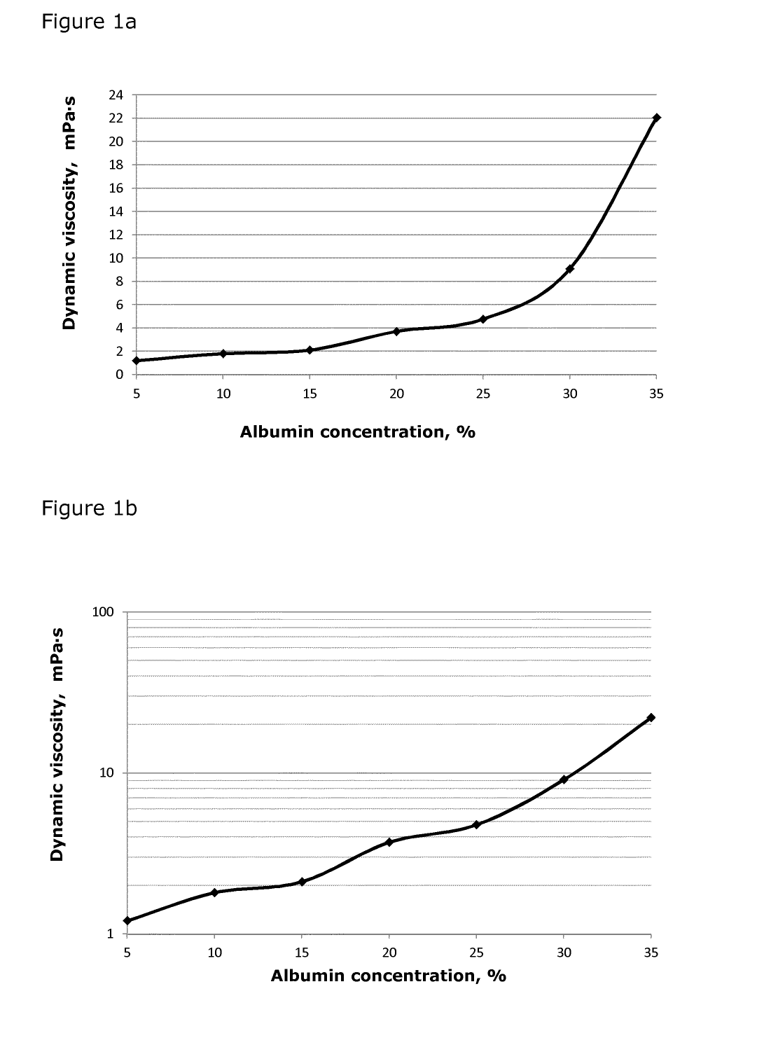

Flow curve analysis was conducted on all prepared samples and the dynamic viscosity was calculated. The viscosity of liquid compositions of albumin from 5 to 35% is presented in Table 2b and FIGS. 1a and 1b.

TABLE 2b

| Albumin | Albumin | | Dynamic |
|---|---|---|---|
| concentration, | (Recombumin ®), | | viscosity, |
| % w/w | g of 35% solution | Added buffer, g | mPa · s |
| 5 | 7.14 | 42.86 | 1.206 |
| 10 | 14.28 | 35.7 | 1.803 |
| 15 | 21.42 | 28.56 | 2.109 |
| 20 | 28.56 | 21.44 | 3.709 |
| 25 | 35.7 | 14.28 | 4.768 |
| 30 | 42.84 | 7.14 | 9.082 |
| 35 | 50 | 0 | 22.06 |

FIG. 1a shows the data of Table 2b with the dynamic viscosity plotted on a linear axis. FIG. 1b shows the data of Table 2b with the dynamic viscosity plotted on a logarithmic axis. These data show that the viscosity of a liquid composition of albumin has an approximately ogarithmic relationship with the concentration of albumin.

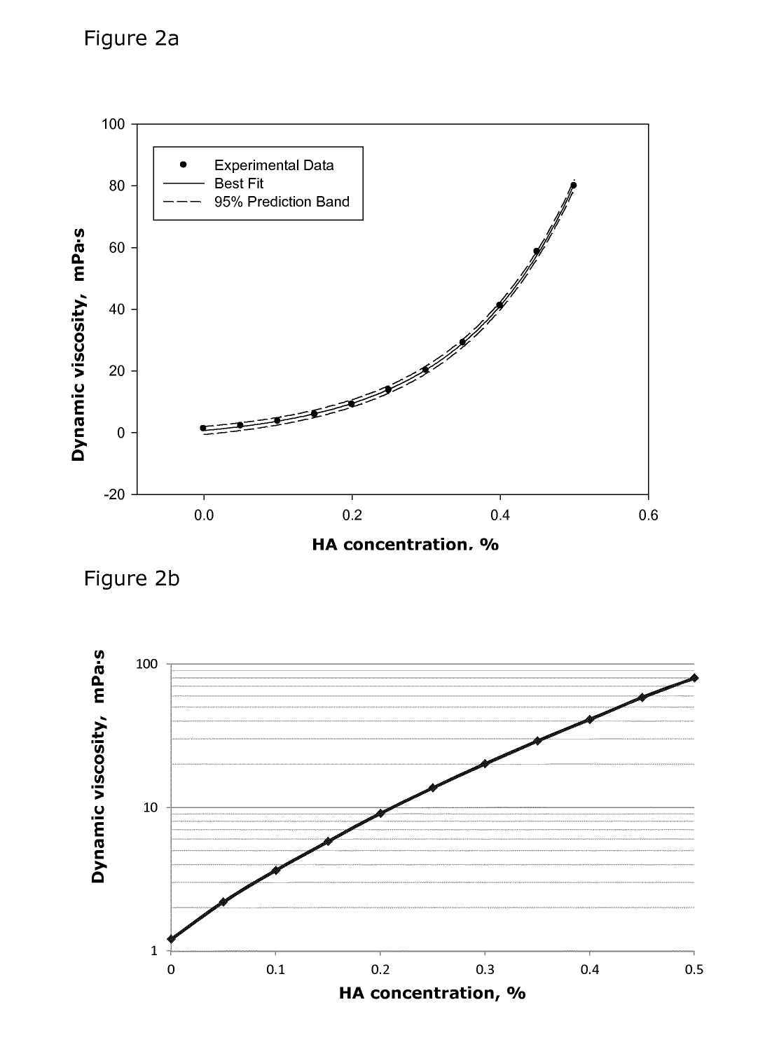

The viscosity of liquid compositions of 5% (w/w) albumin and 0 to 0.5% (w/w) hyaluronic acid (HA) is presented in Table 3 and FIGS. 2a and 2b.

TABLE 3

| Albumin | 850T HA | | |
|---|---|---|---|
| concentration, | concentration, | Added HA powder, | Dynamic |
| % w/w | % w/w | g | viscosity, mPa · s |
| 5 | 0 | 0 | 1.206 |
| 5 | 0.05 | 0.025 | 2.197 |
| 5 | 0.1 | 0.05 | 3.641 |
| 5 | 0.15 | 0.075 | 5.809 |
| 5 | 0.2 | 0.1 | 9.098 |
| 5 | 0.25 | 0.125 | 13.696 |
| 5 | 0.3 | 0.15 | 20.15 |
| 5 | 0.35 | 0.175 | 29.07 |
| 5 | 0.4 | 0.2 | 41.096 |
| 5 | 0.45 | 0.225 | 58.55 |
| 5 | 0.5 | 0.25 | 79.863 |

FIG. 2a shows the data of Table 3 with the dynamic viscosity plotted on a linear axis and also provides the curve of best fit and the upper and lower 95% prediction intervals. FIG. 2b shows the data of Table 3 with the dynamic viscosity plotted on a logarithmic axis. These data show that the viscosity of a liquid composition of 5% albumin containing HA has a very close to logarithmic relationship with the concentration of HA. The data of Table 3 were inputted into SigmaPlot version 11 (build 11.0.0.75, Systat Software Inc., San Jose Calif. USA) and the curve fitted using single, 3 parameter exponential growth (y=y0+a*EXP(b*x)) to derive best fit line and the 95% prediction interval. The prediction interval is the region of uncertainties in predicting the response for a single additional observation. This resulted in:

Curve of best fit: y=−2.658+3.348*EXP(6.423*x)

Lower prediction interval: y=−3.943+3.383*EXP(6.399*x)

Upper prediction interval: y=−1.374+3.314*EXP(6.446*x)

In these equations, y is dynamic viscosity (mPa·s) and x is HA concentration (% w/w).

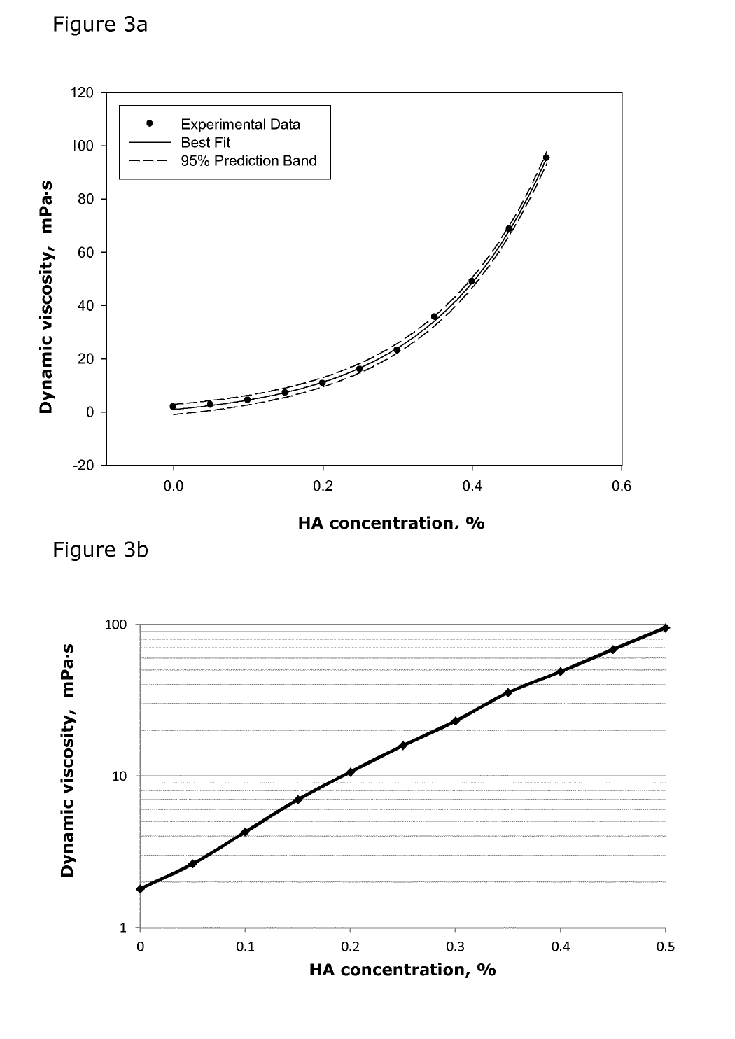

The viscosity of liquid compositions of 10% (w/w) albumin and 0 to 0.5% (w/w) of hyaluronic acid (HA) with an average molecular weight of 850 KDa is presented in Table 4 and FIGS. 3a and 3b.

TABLE 4

| Albumin | 850T HA | | |
|---|---|---|---|
| concentration, | concentration, | Added HA powder, | Dynamic |
| % w/w | % w/w | g | viscosity, mPa · s |
| 10 | 0 | 0 | 1.803 |
| 10 | 0.05 | 0.025 | 2.638 |
| 10 | 0.1 | 0.05 | 4.278 |
| 10 | 0.15 | 0.075 | 6.997 |
| 10 | 0.2 | 0.1 | 10.644 |
| 10 | 0.25 | 0.125 | 15.904 |
| 10 | 0.3 | 0.15 | 23.084 |
| 10 | 0.35 | 0.175 | 35.506 |
| 10 | 0.4 | 0.2 | 48.863 |
| 10 | 0.45 | 0.225 | 68.518 |
| 10 | 0.5 | 0.25 | 95.253 |

FIG. 3a shows the data of Table 4 with the dynamic viscosity plotted on a linear axis and also provides the curve of best fit and the upper and lower 95% prediction intervals. FIG. 3b shows the data of Table 4 with the dynamic viscosity plotted on a logarithmic axis. These data show that the viscosity of a liquid composition of 10% albumin containing HA has a very close to logarithmic relationship with the concentration of HA. The data of Table 4 were inputted into SigmaPlot version 11 (build 11.0.0.75, Systat Software Inc., San Jose Calif. USA) and the curve fitted using single, 3 parameter exponential growth (y=y0+a*EXP(b*x)) to derive best fit line and the 95% prediction interval. The prediction interval is the region of uncertainties in predicting the response for a single additional observation. This resulted in:

Curve of best fit: y=−2.853+3.815*EXP(6.501*x)

Lower prediction interval: y=−4.736+3.864*EXP(6.472*x)

Upper prediction interval: y=−0.971+3.768*EXP(6.531*x)

In these equations, y is dynamic viscosity (mPa·s) and x is HA concentration (% w/w).

Therefore, the data of FIGS. 2 and 3 show that the viscosity of a liquid composition of albumin can be controlled by adding a viscosity modifier. Furthermore, the data show that the amount of viscosity modifier required to achieve a desired viscosity can be calculated.

### Example 2

The viscous properties of liquid formulations comprising albumin and/or hydroxyl propyl methyl cellulose (HPMC) were studied.

Materials: Recombinant human serum albumin: 10% (w/w) preparation of AIblX (Novozymes Biopharma DK A/S); Sodium chloride; Deionized water (MilliQ water, freshly tapped); HPMC: Sigma Aldrich, 2600-5600 cP (2% solution in water at 20° C.).

Equipment: Viscosity was measured using a rheometer (Anton Paar Physica MCR301) at 25° C. using Geometry; C-CC27-T200/SS, CC27.

Liquid compositions were prepared by mixing the required amounts of albumin and/or hyaluronic acid in the following buffer: Buffer: 3000 mL; Sodium Chloride—250 mM (58.44 g/mol); MilliQ water—q.s.

TABLE 5

| Component for 3000 g | Amount, g | |
|---|---|---|
| Sodium Chloride | 43.83 | |
| MilliQ water (to volume) | (to volume) 3000.0 | g |

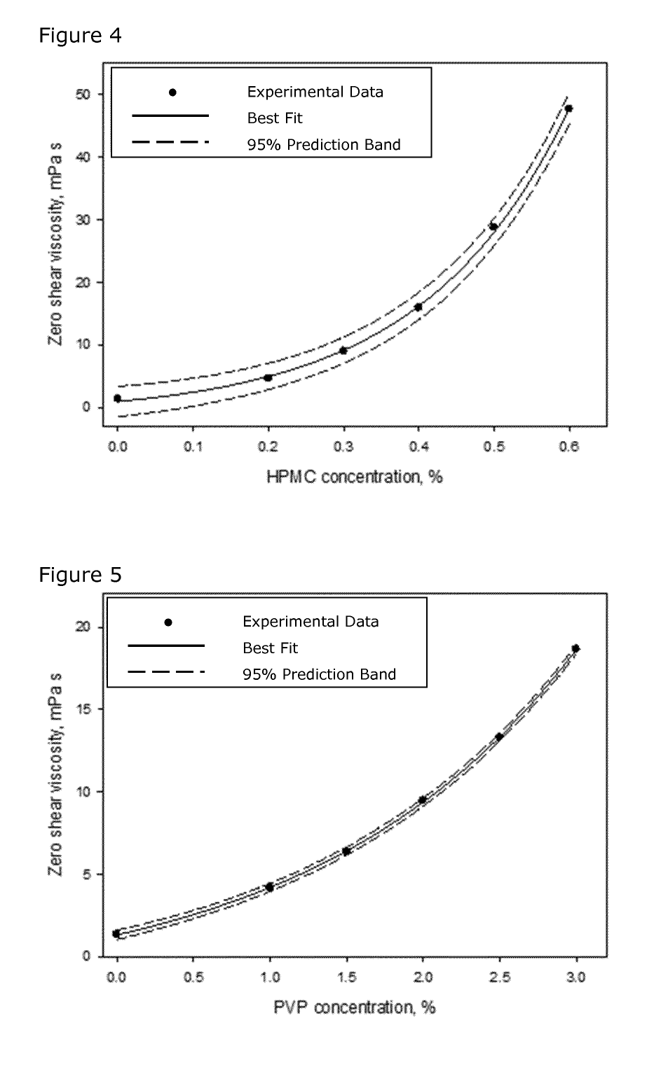

The viscosity of liquid compositions of 5% (w/w) albumin and 0 to 0.6% (w/w) HPMC is presented in Table 6 and FIG. 4.

TABLE 6

| Albumin | HPMC | | |
|---|---|---|---|
| concentration, | concentration, | Added HPMC | Dynamic viscosity, |
| % w/w | % w/w | powder, g | mPa · s |
| 5 | 0 | 0 | 1.33 |
| 5 | 0.2 | 0.1 | 4.55 |
| 5 | 0.3 | 0.15 | 8.91 |
| 5 | 0.4 | 0.2 | 15.9 |
| 5 | 0.5 | 0.25 | 28.75 |
| 5 | 0.6 | 0.3 | 47.65 |

FIG. 4 shows the data of Table 6 with the dynamic viscosity plotted on a linear axis and also provides the curve of best fit and the upper and lower 95% prediction intervals. These data show that the viscosity of a liquid composition of 5% albumin containing HPMC has a very close to logarithmic relationship with the concentration of HPMC. The data of Table 3 were inputted into SigmaPlot version 11 (build 11.0.0.75, Systat Software Inc., San Jose Calif. USA) and the curve fitted using single, 3 parameter exponential growth (y=y0+a*EXP(b*x)) to derive best fit line and the 95% prediction interval. The prediction interval is the region of uncertainties in predicting the response for a single additional observation. This resulted in:

Curve of best fit: y=−1.3028+2.2132*EXP(5.1690*x)

Lower prediction interval: y=−3.6984+2.3290*EXP(5.0815*x)

Upper prediction interval: y=1.0836+2.1045*EXP(5.2558*x)

In these equations, y is dynamic viscosity (mPa·s) and x is HPMC concentration (% w/w).

The determination of the viscosity of liquid compositions of 10% (w/w) albumin and 0 to 0.6% (w/w) HPMC was attempted. However, at these concentrations a solution was not formed.

Therefore, the data in FIG. 4 show that the viscosity of a liquid composition of albumin can be controlled by adding a viscosity modifier. Furthermore, the data show that the amount of viscosity modifier required to achieve a desired viscosity can be calculated.

### Example 3

The viscous properties of liquid formulations comprising albumin and/or polyvinylpyrrolidone (PVP) were studied.

Materials: Recombinant human serum albumin: 10% (w/w) preparation of AlblX (Novozymes Biopharma DK A/S); Sodium chloride; Deionized water (MilliQ water, freshly tapped); PVP, Sigma Aldrich 360 000 Mw (Fikentscher K-value=80 to 100).

Equipment: Viscosity was measured using a rheometer (Anton Paar Physica MCR301) at 25° C. using Geometry; C-CC27-T200/SS, CC27.

Liquid compositions were prepared by mixing the required amounts of albumin and/or hyaluronic acid in the following buffer: Buffer: 3000 mL; Sodium Chloride—250 mM (58.44 g/mol); MilliQ water—q.s.

TABLE 7

| Component for 3000 g | Amount, g | |
|---|---|---|
| Sodium Chloride | 43.83 | |
| MilliQ water (to volume) | (to volume) 3000.0 | g |

The viscosity of liquid compositions of 5% (w/w) albumin and 0 to 3% (w/w) PVP is presented in Table 8 and FIG. 5.

TABLE 8

| Albumin concentration, | PVP, | Added PVP | Dynamic viscosity, |
|---|---|---|---|
| % w/w | % w/w | powder, g | mPa · s |
| 5 | 0 | 0 | 1.33 |
| 5 | 1.0 | 0.5 | 4.19 |
| 5 | 1.5 | 0.75 | 6.35 |
| 5 | 2.0 | 1 | 9.46 |
| 5 | 2.5 | 1.25 | 13.3 |
| 5 | 3.0 | 1.5 | 18.65 |

FIG. 5 shows the data of Table 8 with the dynamic viscosity plotted on a linear axis and also provides the curve of best fit and the upper and lower 95% prediction intervals. These data show that the viscosity of a liquid composition of 5% albumin containing PVP has a very close to logarithmic relationship with the concentration of PVP. The data of Table 8 were inputted into SigmaPlot version 11 (as described for Example 2). This resulted in:

Curve of best fit: y=−2.3149+3.6315*EXP(0.5844*x)

Lower prediction interval: y=−2.6554+3.7042*EXP(0.5788*x)

Upper prediction interval: y=−1.9763+3.5605*EXP(0.5899*x)

In these equations, y is dynamic viscosity (mPa·s) and x is PVP concentration (% w/w).

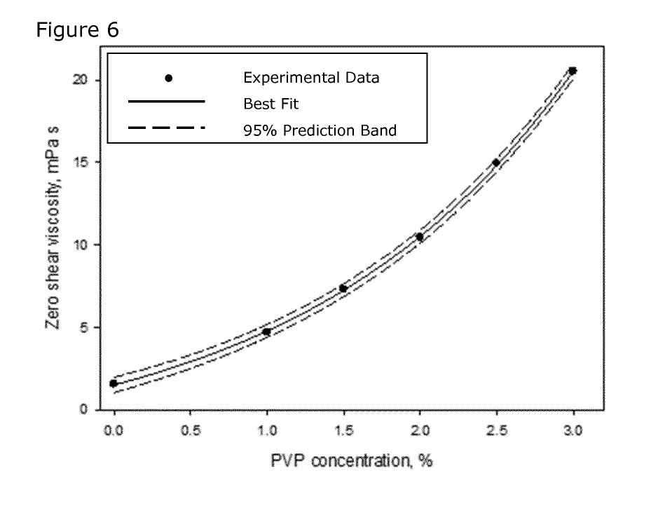

The viscosity of liquid compositions of 10% (w/w) albumin and 0 to 3% (w/w) PVP is presented in Table 9 and FIG. 6.

TABLE 9

| Albumin | | | |
|---|---|---|---|
| concentration, | PVP concentration, | Added PVP | Dynamic viscosity, |
| % w/w | % w/w | powder, g | mPa · s |
| 10 | 0 | 0 | 1.803 |
| 10 | 1.0 | 0.5 | 2.638 |
| 10 | 1.5 | 0.75 | 4.278 |
| 10 | 2.0 | 1 | 6.997 |
| 10 | 2.5 | 1.25 | 10.644 |
| 10 | 3.0 | 1.5 | 15.904 |

FIG. 6 shows the data of Table 9 with the dynamic viscosity plotted on a linear axis and also provides the curve of best fit and the upper and lower 95% prediction intervals. These data show that the viscosity of a liquid composition of 10% albumin containing PVP has a very close to logarithmic relationship with the concentration of PVP. The data of Table 9 were inputted into SigmaPlot version 11 (as described in Example 2). This resulted in:

Curve of best fit: y=−2.8401+4.3501*EXP(0.5607*x)

Lower prediction interval: y=−3.4081+4.4819*EXP(0.5525*x)

Upper prediction interval: y=−2.2770+4.2230*EXP(0.5689*x)

In these equations, y is dynamic viscosity (mPa·s) and x is PVP concentration (% w/w).

Therefore, the data of FIGS. 5 and 6 show that the viscosity of a liquid composition of albumin can be controlled by adding a viscosity modifier. Furthermore, the data show that the amount of viscosity modifier required to achieve a desired viscosity can be calculated.

1. A liquid composition comprising albumin and a viscosity modifier wherein the composition has a dynamic viscosity from 1 to 250 mPa·s at 25° C.

2. A composition according to claim 1 wherein the composition has a dynamic viscosity of from 5 to 250 mPa·s at 25° C.

3. A composition according to claim 1 wherein the composition has a dynamic viscosity of from 40 to 160 mPa·s at 25° C.

4. The composition according to claim 1 wherein albumin is present at from 5 to 25% (w/w).

5. The composition according to claim 1 wherein the viscosity modifier is present at from 0.01 to 10% (w/w).

6. The composition according to claim 1 wherein albumin is present at from 1 to 20% (w/w).

7. The composition according to claim 1 wherein the viscosity modifier is present at from 0.05 to 5% (w/w).

8. The composition according to claim 1 wherein albumin is present at from 5 to 15% (w/w).

9. The composition according to claim 1 wherein the viscosity modifier is present at from 0.05% to 0.5% (w/w).

10. The composition according to claim 1 wherein the viscosity modifier is selected from glycosaminoglycans, extracellular matrix extracts, polyamino acids, gelatin, amylopectin, maltodextrin, dextran, glycogen, chondroitin, cellulose, cellulose derivatives, cyclodextrins, polyesters, polyorthoesters, sulfate, dermatan sulfate, hydrophobic polymers, polyethylene glycols, polyethyleneoxide, polysaccharides, carbomers, polyvinyl alcohols.

11. The composition according to claim 10 wherein the viscosity modifier is an anionic non-sulfated glycosaminoglycan such as hyaluronic acid.

12. The composition according to claim 10 wherein the composition comprises about 5% albumin and the HA concentration and dynamic viscosity of the composition lie on or within the following boundaries:
lower boundary: y=−3.943+3.383*EXP(6.399*x)
upper boundary: y=−1.374+3.314*EXP(6.446*x)
wherein y=dynamic viscosity in mPa·s and x=concentration (% w/w) of HA with an average molecular weight of from about 600 to 1100, such as about 850 KDa.

13. The composition according to claim 10 wherein the composition comprises about 10% albumin and the HA concentration and dynamic viscosity of the composition lie on or within the following boundaries:
lower boundary: y=−4.736+3.864*EXP(6.472*x)
upper boundary: y=−0.971+3.768*EXP(6.531*x)
wherein y=dynamic viscosity in mPa·s and x=concentration (% w/w) of HA with an average molecular weight of from about 600 to 1100, such as about 850 KDa.

14. The composition according to claim 10 wherein the viscosity modifier is HPMC and the composition comprises about 5% albumin and the HPMC concentration and dynamic viscosity of the composition lie on or within the following boundaries:
lower boundary: y=−3.6984+2.23290*EXP(5.0815*x)
upper boundary: y=1.0836+2.1045*EXP(5.2558*x)
wherein y=dynamic viscosity in mPa·s and x=concentration (% w/w) of HPMC.

15. The composition according to claim 10 wherein the viscosity modifier is PVP and the composition comprises about 5% albumin and the PVP concentration and dynamic viscosity of the composition lie on or within the following boundaries:
lower boundary: y=−2.6554+3.7042*EXP(0.5788*x)
upper boundary: y=−1.9763+3.5605*EXP(0.5899*x)
wherein y=dynamic viscosity in mPa·s and x=concentration (% w/w) of PVP.

16. The composition according to claim 10 wherein the viscosity modifier is PVP and the composition comprises about 10% albumin and the PVP concentration and dynamic viscosity of the composition lie on or within the following boundaries:
lower boundary: y=−3.4081+4.4819*EXP(0.5525*x)
upper boundary: y=−2.2770+4.2230*EXP(0.5689*x)
wherein y=dynamic viscosity in mPa·s and x=concentration (% w/w) of PVP.

17. A composition according to claim 1 which does not contain a pharmacologically active compound.

18. A placebo comprising a composition according to claim 1.

19. A method for preparing a composition of a desired viscosity comprising suspending albumin and a viscosity modifier in a liquid in a ratio complying with 1 part viscosity modifier and from 5 to 2500 parts albumin.

20. The method according to claim 19 wherein the composition comprises about 5% albumin and the HA concentration and dynamic viscosity of the composition lie on or within the following boundaries:
lower boundary: y=−3.943+3.383*EXP(6.399*x)
upper boundary: y=−1.374+3.314*EXP(6.446*x)
wherein y=dynamic viscosity in mPa·s and x=concentration (% w/w) of HA with an average molecular weight of from about 600 to 1100, such as about 850 KDa.

21. The method according to claim 19 wherein the composition comprises about 10% albumin and the HA concentration and dynamic viscosity of the composition lie on or within the following boundaries:
lower boundary: y=−4.736+3.864*EXP(6.472*x)
upper boundary: y=−0.971+3.768*EXP(6.531*x)
wherein y=dynamic viscosity in mPa·s and x=concentration (% w/w) of HA with an average molecular weight of from about 600 to 1100, such as about 850 KDa.

22. The method according to claim 19 wherein the viscosity modifier is a cellulose or cellulose derivative and the composition comprises about 5% albumin and the HPMC concentration and dynamic viscosity of the composition lie on or within the following boundaries:
lower boundary: y=−3.6984+2.23290*EXP(5.0815*x)
upper boundary: y==1.0836+2.1045*EXP(5.2558*x)
wherein y=dynamic viscosity in mPa·s and x=concentration (% w/w) of HPMC.

23. The method according to claim 19 wherein the viscosity modifier is PVP and the composition comprises about 5% albumin and the PVP concentration and dynamic viscosity of the composition lie on or within the following boundaries:
lower boundary: y=−2.6554+3.7042*EXP(0.5788*x)
upper boundary: y=−1.9763+3.5605*EXP(0.5899*x)
wherein y=dynamic viscosity in mPa·s and x=concentration (% w/w) of PVP.

24. The method according to claim 19 wherein the viscosity modifier is PVP and the composition comprises about 10% albumin and the PVP concentration and dynamic viscosity of the composition lie on or within the following boundaries:
lower boundary: y=−3.4081+4.4819*EXP(0.5525*x)
upper boundary: y=−2.2770+4.2230*EXP(0.5689*x)
wherein y=dynamic viscosity in mPa·s and x=concentration (% w/w) of PVP.

25. (canceled)

26. A method of administering to a patient or a method of carrying out a clinical trial comprising:
(a) administering to a first group of patients, a pharmaceutical test composition;
(b) administering to a second group of patients, a placebo which has a viscosity from 50 to 200 of the viscosity of the pharmaceutical test composition, wherein the placebo is a composition according to claim 1 or produced according to the method of claim 19;
(c) analyzing the effectiveness of the pharmaceutical test composition relative to the placebo.


SEQUENCE LISTING (2 sequences)

SEQ ID NO: 1 - DNA, 1758 bp, Homo sapiens
  misc_feature (1)..(1758): cDNA encoding HSA
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 2 - protein, 585 aa, Homo sapiens [sequence omitted]